(19) United States
(12) Patent Application Publication
(10) Pub. No.: US 2016/0192823 A1
(43) Pub. Date: Jul. 7, 2016

(54) ENDOSCOPE SYSTEM

(71) Applicant: OLYMPUS CORPORATION, Tokyo (JP)

(72) Inventors: Koji YASUNAGA, Tokyo (JP); Ai YONEKURA, Tokyo (JP)

(73) Assignee: OLYMPUS CORPORATION, Tokyo (JP)

(21) Appl. No.: 15/071,293

(22) Filed: Mar. 16, 2016

Related U.S. Application Data
(63) Continuation of application No. PCT/JP2015/056538, filed on Mar. 5, 2015.

(30) Foreign Application Priority Data
Aug. 11, 2014 (JP) 2014-163652

(51) Int. Cl. A61B 1/005; A61B 1/05; A61B 1/00

(52) U.S. Cl. CPC A61B 1/0052; A61B 1/00009; A61B 1/00045; A61B 1/05

(57) ABSTRACT

An endoscope system of the present invention includes an image pickup section that photoelectrically converts an optical image to generate an electronic image signal, an insertion portion that incorporates the image pickup section, includes a bendable bending portion and is inserted into a subject, a lever portion that is swingable in a direction orthogonal to a central axis, held so as to be rotatable around the central axis and swung to thereby bend the bending portion, and a video processor section that receives an image signal generated by the image pickup section, generates a display image signal, performs rotating image signal processing on the display image signal in accordance with a rotation of the lever portion and outputs the processing result to an image display apparatus.

### CROSS REFERENCE TO RELATED APPLICATION

This application is a continuation application of PCT/JP2015/056538 filed on Mar. 5, 2015 and claims benefit of Japanese Application No. 2014-163652 filed in Japan on Aug. 11, 2014, the entire contents of which are incorporated herein by this reference.

### BACKGROUND OF THE INVENTION

1. Field of the Invention

The present invention relates to an endoscope system constructed of an endoscope provided with an image pickup section and a bending portion on a distal end side of an insertion portion and an operation section provided with a bending operation member on a proximal end side of the insertion portion, and an image display apparatus or the like that displays an image acquired by the image pickup section.

2. Description of the Related Art

In recent years, endoscope systems for observing an inside of a body cavity or an inside of an apparatus are widely used in a medical field or an industrial field. In the medical field in particular, various forms of endoscope systems are being put to practical use such as endoscopes suitable for use in inserting an insertion portion from an opening of an organ such as the oral cavity or the anus to observe an inside of a digestive organ such as the esophagus, the stomach, the large intestine or apply various treatments or laparoscopes suitable for use in inserting an insertion portion from a hole opened on the body surface of the abdomen to observe an inside of the abdominal cavity or apply treatment such as surgery.

Such an endoscope system is constructed of an endoscope that includes an image pickup section and a bending portion provided on a distal end side of an elongated insertion portion and bends the bending portion by swinging a bending operation member provided on the operation section on a proximal end side of the insertion portion by hand, finger or the like to thereby orient an observation field of view of the image pickup section toward a desired direction, a video processor that performs various types of image signal processing based on electronic image signals acquired by the image pickup section and an image display apparatus or the like that receives an image signal to be displayed generated by the video processor and displays an image.

In conventional endoscope systems, as a bending operation member provided in the operation section, there is not only a rotating member or a rotation lever member that bends the bending portion by, for example, rotating the bending portion around the axis but also a so-called joystick type lever member that bends the bending portion by swinging the bending portion in a direction orthogonal to the axis.

Furthermore, in conventional endoscope systems, laparoscopes provided with a rigid endoscope including a cylindrical rigid insertion portion are generally used for laparoscopic surgery, for example. However, in recent years, a variety of bending type laparoscopes provided with a bending portion on a distal end side of the insertion portion are being proposed and generally put to practical use.

In such a conventional bending type laparoscope, when the bending portion is bent in a vertical direction and horizontal direction compositely, an image pickup plane of the image pickup section provided inside the distal end portion may be rotated with respect to an observation plane of an object to be observed. Therefore, during a laparoscopic surgery, for example, even when a scopist (endoscopic surgery assistant; so-called cameraman) holds the operation section by adjusting its vertical direction appropriately, an observed image displayed on a display screen of the image display apparatus may be rotated or inclined with respect to the display screen depending on a bending operation condition of the distal end portion.

A rotation or inclination or the like of a display image that may occur against the user's will may not matter in the case of an endoscope for digestive organs, for example, but in the case of a laparoscope, since the vertical direction of the display image or image direction relative to the user (surgeon) is considered important, the rotation or inclination or the like of the display image may cause a problem of affecting the user's (surgeon's) treatment operability (hand-eye coordination; cooperativeness of manipulation with respect to visual information) or comprehension of anatomy or the like. Furthermore, the user (surgeon) may want to intentionally rotate the top-and-bottom direction of an image during an operation.

Thus, various configurations are conventionally proposed in, for example, Republication of PCT International Publication No. WO2011/024565, Japanese Patent No. 4365860 or the like in which the endoscope distal end portion is rotated, the image pickup plane of the image pickup section provided inside the distal end portion is rotated with respect to the observation target plane, and the display image is thereby rotated and corrected.

Republication of PCT International Publication No. WO2011/024565 or the like discloses an endoscope system configured to apply a joystick type lever member as a bending operation member and includes an operation button to cause the bending portion to rotate around the insertion axis in addition to the bending operation member.

Furthermore, above Japanese Patent No. 4365860 or the like discloses an endoscope system configured to apply a rotation lever member as a bending operation member and include an operation button for rotating a display image in addition to the bending operation member.

### SUMMARY OF THE INVENTION

An endoscope system according to an aspect of the present invention includes an image pickup section that photoelectrically converts an optical image to generate an electronic image signal, an insertion portion that incorporates the image pickup section, includes a bendable bending portion and is inserted into a subject, an operation portion connected to a proximal end side of the insertion portion, a lever portion that is provided in the operation portion, connected to the bending portion via a towing member, swingable in a direction orthogonal to a central axis, held so as to be rotatable around the central axis and swung to thereby bend the bending portion, and a video processor section that receives an image signal generated by the image pickup section, generates a display image signal, performs rotating image signal processing on the display image signal in accordance with a rotation of the lever portion and outputs a processing result to an image display apparatus.

### BRIEF DESCRIPTION OF THE DRAWINGS

FIG. 1 is a configuration diagram schematically illustrating an overall configuration of an endoscope system according to a first embodiment of the present invention;

FIG. 2 is a configuration diagram mainly illustrating a bending operation unit inside an operation portion of the endoscope system in FIG. 1;

FIG. 3 is a configuration diagram illustrating a schematic configuration of an endoscope system and a bending operation unit inside an operation portion according to a second embodiment of the present invention;

FIG. 4 is an enlarged exploded perspective view of main parts with some of components of the bending operation unit in the endoscope system in FIG. 3 extracted and shown in exploded form;

FIG. 5 is an external perspective view illustrating an entire endoscope in an endoscope system according to a third embodiment of the present invention;

FIG. 6 is an external perspective view illustrating an image pickup section extracted from the endoscope of the endoscope system in FIG. 5;

FIG. 7 is an enlarged cross-sectional view of main parts illustrating an inner structure of a distal end portion in the endoscope of the endoscope system in FIG. 5 and a configuration of the image pickup section in particular;

FIG. 8 is a configuration diagram illustrating a schematic configuration of the endoscope system in FIG. 5 and a bending operation unit inside the operation portion; and

FIG. 9 is a configuration diagram illustrating a schematic configuration of an endoscope system and a bending operation unit inside an operation portion according to a fourth embodiment of the present invention.

### DETAILED DESCRIPTION OF THE PREFERRED EMBODIMENTS

Hereinafter, the present invention will be described according to embodiments illustrated in the accompanying drawings. The drawings used for the following description are schematically illustrated, and dimensional relationships among respective members and their scales or the like may be differentiated for each component so as to be illustrated to such an extent that each component is recognizable in the drawings. Therefore, the present invention is not limited to only configurations illustrated in the drawings with regard to quantities of components, shapes of the components, size ratios among the components and relative positional relationships among the components or the like described in the drawings.

### First Embodiment

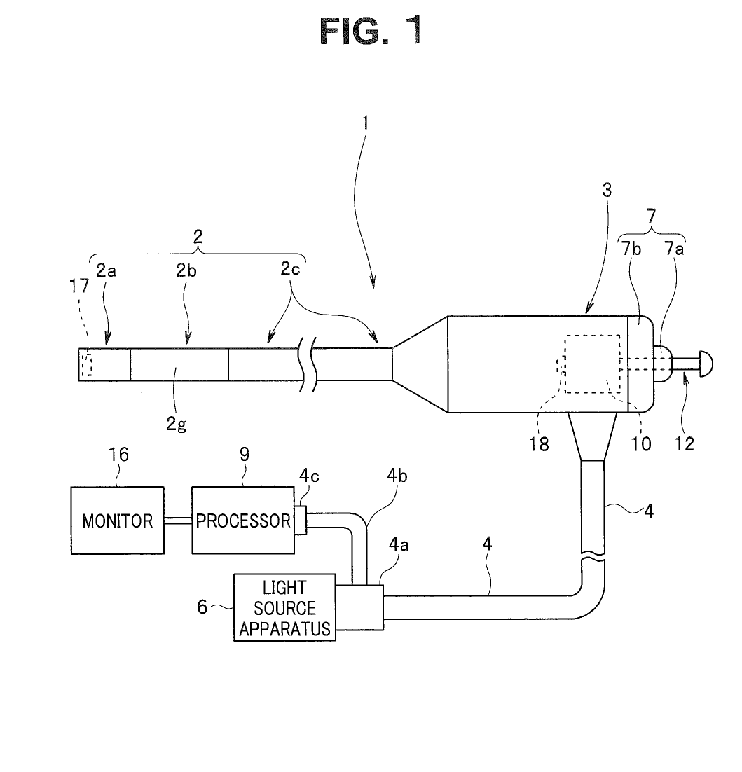

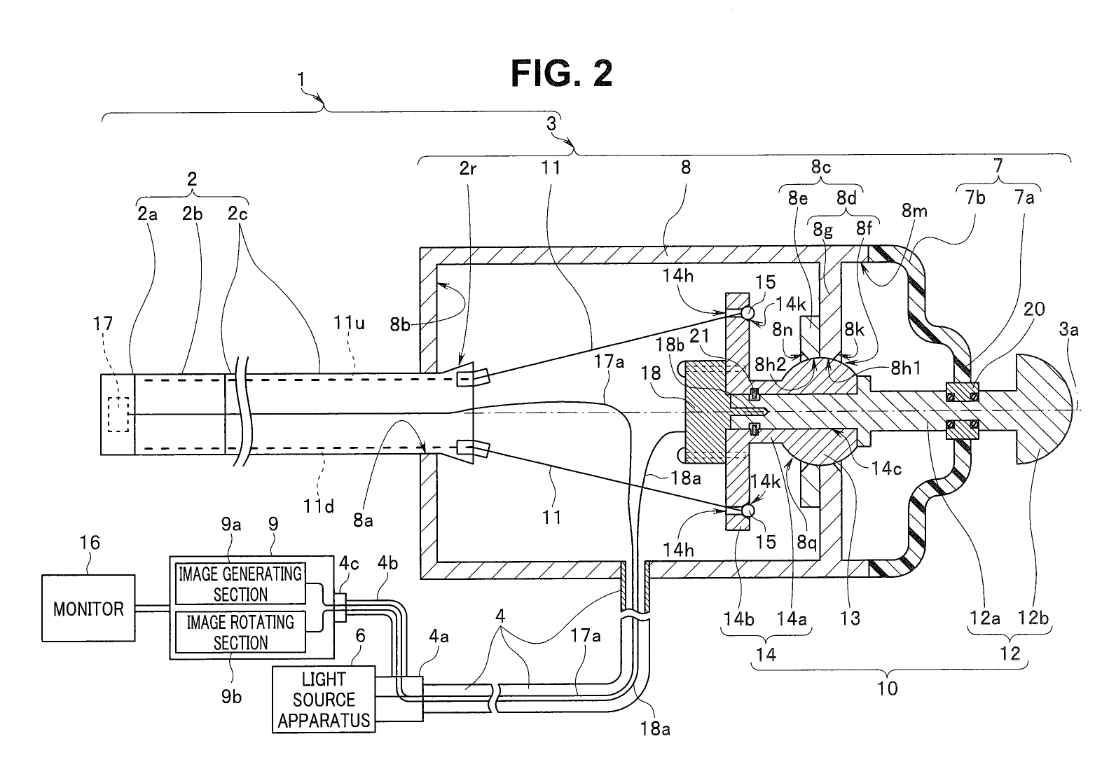

First, a schematic configuration of an endoscope system according to a first embodiment of the present invention will be described below using mainly FIG. 1. FIG. 1 is a diagram schematically illustrating an overall configuration of the endoscope system according to the first embodiment of the present invention. FIG. 2 is a configuration diagram mainly illustrating a bending operation unit inside an operation portion of the endoscope system in FIG. 1.

The endoscope system of the present embodiment is configured by including an endoscope 1, various external devices connected to the endoscope 1 such as a light source apparatus 6 and a video processor section 9 which is a signal control processing apparatus as shown in FIG. 1.

Here, the light source apparatus 6 is provided to emit illuminating light onto an object to be observed from a front end face of the endoscope 1 using the endoscope 1. A detailed configuration applied thereto is similar to that of a conventional endoscope.

The video processor section 9 is a circuit section configured by including a control circuit responsible for overall control of the present endoscope system and a signal circuit that receives an image signal generated by an image pickup section (17; which will be described later) and applies various kinds of image signal processing such as processing of generating a display image signal. A monitor apparatus 16 (simply displayed as a monitor in FIG. 1) which is an image display apparatus is connected to the video processor section 9. In this way, an image signal generated by the image pickup section 17 and processed by applying various kinds of processing (processing result) thereto is outputted to the monitor apparatus 16 and an image corresponding to the image signal is configured to be displayed so as to be viewable on a display screen of the monitor apparatus 16.

The endoscope 1 is constructed of an elongated tube-like insertion portion 2, an operation portion 3 connected on a proximal end side of the insertion portion 2 and a universal cord 4 that extends from the operation portion 3, or the like.

An LG connector 4a connected to the light source apparatus 6 which is an external device is provided at a distal end portion of the universal cord 4. One end of an illumination light guide (not shown; see reference numeral 17a in FIG. 2) for transmitting illuminating light emitted from the light source apparatus 6 to a distal end side of the insertion portion 2 of the endoscope 1 is connected to the LG connector 4a. The illumination light guide (not shown; 17a) is passed from the LG connector 4a through the universal cord 4, the operation portion 3 and the insertion portion 2 respectively and the other end thereof is disposed behind an illumination optical system (not shown) provided on a front end face of the insertion portion 2. In this configuration, illuminating light emitted from the light source apparatus 6 is guided from the LG connector 4a connected to the light source apparatus 6 via the illumination light guide (not shown; 17a) and emitted forward from the front end face of the insertion portion 2.

Furthermore, a cable 4b that electrically connects the LG connector 4a and the video processor section 9 extends from one side of the LG connector 4a. A signal connector 4c is provided at a distal end portion of the cable 4b. Signal cables (not shown; see reference numerals 17a and 18a in FIG. 2) for electrically connecting between the video processor section 9 and electrical components inside the operation portion 3 (e.g., rotation angle detection section 18 which will be described later) and an electrical configuration unit inside the distal end of the insertion portion 2 (e.g., image pickup section 17 which will be described later) and securing exchange of various electric signals (control signal, image pickup signal, detection signal or the like) are connected to the signal connector 4c. The signal cable (not shown; 17a) is passed from the signal connector 4c through the universal cord 4, the operation portion 3 and the insertion portion 2 respectively and the other end thereof is connected to a predetermined component as appropriate. This configuration allows a control signal from the video processor section 9 to be transmitted to the image pickup section 17 inside the distal end of the insertion portion 2 to drive the image pickup section 17 or allows an output signal from the image pickup section 17 and a detection signal of the rotation angle detection section 18 inside the operation portion 3 to be transmitted to the video processor section 9.

The insertion portion 2 in the endoscope 1 is a portion inserted into the body cavity and configured by consecutively connecting a distal end portion 2a, a bending portion 2b and a rigid tube portion 2c in that order from the distal end side.

The distal end portion 2a is made of a rigid material such as stainless steel. The image pickup section 17 is disposed inside the distal end portion 2a, which is configured by including an image pickup optical system that causes an optical image of an object to be observed to be formed and an image pickup device that receives the optical image formed by the image pickup optical system, photoelectrically converts the optical image to generate an electronic image signal, or the like. A signal cable (not shown; 17a) extends from the image pickup section 17.

The bending portion 2b is a component configured to be bendable in four directions of up, down, right and left, for example, with respect to an insertion axis direction. The bending portion 2b is mainly constructed of a bending piece unit (not shown) which is configured to be freely bendable and bending rubber 2g that covers an outer surface of the bending piece unit. The bending piece unit (not shown) is a unit configured to pivotably connect a plurality of bending pieces so that it can bend up, down, right and left. The bending rubber 2g has a predetermined elastic force and coats an outer surface of the bending piece unit. Note that since the configuration of the bending portion 2b itself is a portion not directly related to the present invention, detailed description thereof is omitted assuming that it has a configuration similar to that of a conventional endoscope.

Furthermore, a configuration in which the bending portion 2b is caused to bend in an up-down direction is substantially the same as a configuration in which the bending portion 2b is caused to bend in a right-left direction. For this reason, the configuration in which the bending portion 2b is caused to bend in the up-down direction will be mainly described below. Furthermore, the bending portion 2b is not limited to the mode in which the bending portion 2b can be bent in four directions of up, down, right and left as described above, but other modes may also be available if, for example, the bending portion 2b can be bent in at least two directions with respect to the insertion axis direction.

The rigid tube portion 2c is a metal member such as stainless steel formed into a tubular shape.

An air feeding tube and a water feeding tube or the like as well as the above-described signal cable and illumination light guide (see reference numeral 17a in FIG. 2) are inserted into the insertion portion 2.

Note that a so-called rigid insertion portion configured by consecutively connecting the distal end portion 2a, the bending portion 2b and the rigid tube portion 2c is illustrated as an example of the insertion portion 2 in the endoscope system of the present embodiment. However, the mode of the insertion portion 2 to which the present invention is applicable is not limited to the rigid insertion portion. The present invention is likewise applicable to a flexible insertion portion configured by consecutively connecting the distal end portion 2a, the bending portion 2b and a flexible tube portion having flexibility (not shown) as well. The present invention is likewise applicable to a mode of the insertion portion in which a treatment instrument channel tube or the like is inserted into the insertion portion 2 as well.

In the endoscope 1, the operation portion 3 has a plurality of operation members including such as an angle lever 12 which is a bending operation member for performing a bending operation and a lever portion provided on an outer surface side and is mainly constructed of an external housing that incorporates various components therein (e.g., bending operation unit 10 including such as a rotation angle detection section 18, details of which will be described later) and a rubber boot 7 which is an external member disposed so as to cover a proximal end side of the external housing.

The rubber boot 7 is an elastic member having predetermined resilience and is formed of an elastic holding portion 7a and an elastic fixing portion 7b. The elastic holding portion 7a is a region having a function of elastically holding the angle lever 12. The elastic fixing portion 7b is a region that functions as a closing member disposed so as to cover an opening 8m (see FIG. 2) of an inner frame 8 provided inside the external housing of the operation portion 3.

A plurality of operation members are provided on an outer surface of the operation portion 3. Among the operation members, an operation member shown by reference numeral 12 in FIG. 1 is an angle lever which is a bending operation member. Note that in the present embodiment, only the angle lever 12 is illustrated as a member directly related to the present invention, and illustration and description of other operation members which are not related to the present invention are omitted.

The angle lever 12 is a bending operation member for remotely operating the bending portion 2b. A mode of, for example, joystick type is used as the mode of this angle lever 12.

The angle lever 12 is a lever-like member formed so as to protrude outward from the elastic holding portion 7a of the rubber boot 7 of the operation portion 3. A configuration is adopted so that the bending direction in four directions of up, down, right and left and the amount of bending of the bending portion 2b may be freely set by tilting the angle lever 12, and changing and adjusting the tilting direction and the tilting angle as appropriate.

Although a detailed configuration will be described later, this angle lever 12 (lever portion) is a component provided in the operation portion 3. The angle lever 12 is a component connected to the bending portion 2b via a plurality of bending wires (towing members which will be described later; see reference numeral 11 in FIG. 2). The angle lever 12 is a component that is swingable in a direction orthogonal to a central axis (see reference numeral 3a in FIG. 2). The angle lever 12 is a component rotatably held around the central axis (3a). The angle lever 12 is a component that is swung to bend the bending portion 2b.

Note that the angle lever 12 is assumed to be disposed at a position (see FIG. 1) on the proximal end side of the operation portion 3 as shown in FIG. 1 in the present embodiment, but this mode is simply an example, and the position at which the angle lever 12 is disposed is not limited to this mode.

Next, of the internal configuration of the operation portion 3 in the endoscope 1 of the endoscope system according to the present embodiment, a detailed configuration of the bending operation unit in particular will be described using mainly FIG. 2.

The inner frame 8 is disposed inside the external housing of the operation portion 3. Note that FIG. 2 shows the operation portion 3 using only the inner frame 8 while omitting illustration of the external housing.

The inner frame 8 of the operation portion 3 has, for example, a substantially cylindrical shape as a whole and has a substantially circular cross-sectional shape orthogonal to the major axis 3a (see FIG. 2). A closed bottom surface 8b is formed at one end of the inner frame 8 in the major axis direction and the opened opening 8m is formed at the other end thereof in the major axis direction.

An insertion portion disposition portion 8a is formed in the bottom surface 8b, to which a proximal end portion 2r of the insertion portion 2 is fixed. The elastic fixing portion 7b of the rubber boot 7 which is a closing member is fixed to the opening 8m so as to cover the opening 8m and secure water tightness. A bending mechanism attaching portion 8c is formed in the vicinity of the opening 8m and inside the inner frame 8. The bending mechanism attaching portion 8c is constructed of an attaching portion body 8d and a cover portion 8e.

Of these portions, the attaching portion body 8d is constructed of a bending mechanism disposition portion 8f and a frame fixing portion 8g. Of these portions, the frame fixing portion 8g is formed into a substantially disk shape corresponding to a substantially circular cross section orthogonal to the major axis of the inner frame 8 and an outer circumferential edge thereof is fixed to an inner wall surface of the inner frame 8 through, for example, solder bond or using an adhesive or the like.

The bending mechanism disposition portion 8f made up of a substantially circular open hole portion is formed at a substantially central part of the frame fixing portion 8g. A sphere 13 that forms part of a swinging frame 14 of a bending operation unit 10 which will be described later is disposed in the bending mechanism disposition portion 8f. For this reason, the bending mechanism disposition portion 8f is formed by including a first semi-spherical concave portion 8h1 and a tilting undercut 8k. The first semi-spherical concave portion 8h1 is a concave curved surface formed on a circumferential surface on an inner edge of the bending mechanism disposition portion 8f. A curvature of the concave curved surface of the first semi-spherical concave portion 8h1 is formed to be substantially equal to the curvature of the outer surface of the sphere 13. The tilting undercut 8k is a through hole formed so as to have an outwardly tapered surface in the open hole portion of the bending mechanism disposition portion 8f.

On the other hand, the cover portion 8e is formed so as to have a substantially circular open hole portion in a substantially central part as with the attaching portion body 8d and is a member fixedly disposed in a region where the open hole portion thereof matches the open hole portion of the bending mechanism disposition portion 8f. The open hole portion of the cover portion 8e is formed to be substantially equal to the open hole portion of the bending mechanism disposition portion 8f and is formed so as to have a second semi-spherical concave portion 8h2 and a swinging undercut 8n. The second semi-spherical concave portion 8h2 is a concave curved surface formed on a circumferential surface of an inner edge of the open hole portion of the cover portion 8e. A curvature of the concave curved surface of the second semi-spherical concave portion 8h2 is formed to be substantially equal to the curvature of an outer surface of the sphere 13 as in the case of the first semi-spherical concave portion 8h1. The swinging undercut 8n is a through hole formed so as to have an outwardly tapered surface in the open hole portion of the cover portion 8e.

Note that the cover portion 8e is fixed to one flat plane of the attaching portion body 8d by screws so as to be integrated therewith. In this case, a counterbore hole and a thread undercut are formed in the cover portion 8e and a concave portion provided with a female thread is formed in the attaching portion body 8d (details are not shown).

With such a configuration, the bending mechanism attaching portion 8c is formed so as to allow the sphere 13 of the angle lever 12 which will be described later to be sandwiched between the bending mechanism disposition portion 8f and the cover portion 8e of the attaching portion body 8d. In an assembled state thereof, the angle lever 12 is configured to be tiltable around the sphere 13 with respect to the major axis 3a or rotatable around the major axis 3a.

For this reason, when the cover portion 8e is fixed to the bending mechanism disposition portion 8f, the sphere 13 is disposed in the first semi-spherical concave portion 8h1 and then the cover portion 8e is fixed with screws. As a result, the sphere 13 is freely movably disposed in the sphere disposition portion 8q configured by combining the first semi-spherical concave portion 8h1 and the second semi-spherical concave portion 8h2 of the bending mechanism attaching portion 8c in the inner frame 8 along with the operation of the angle lever 12 (details will be described later).

In the operation portion 3, the bending operation unit 10 is disposed inside the inner frame 8. The bending operation unit 10 is mainly constructed of a plurality of bending wires 11 which are towing members, the angle lever 12 which is a bending operation member to add a towing force to the bending wires, the swinging frame 14 which is provided integrally with the angle lever 12 to integrally form the sphere 13 disposed in the sphere disposition portion 8q to lock the respective proximal end portions of the plurality of bending wires 11 and the rotation angle detection section 18 that detects an angle of rotation of the angle lever 12.

A plurality of wires are provided as the bending wires 11, in association with bending directions, for example, four directions of up, down, right and left respectively. FIG. 2 illustrates a bending wire 11u for upward bending and a bending wire 11d for downward bending. A distal end of each bending wire 11 is fixed to a predetermined region of a distal end bending piece (not shown) of the bending piece unit making up the bending portion 2b. Furthermore, a spherical wire locking member 15 is fixed at a proximal end of each bending wire 11.

The angle lever 12 is formed by including, for example, a metallic bar-like lever body 12a and a semi-spherical finger hooking portion 12b. The finger hooking portion 12b is integrally fixed to one end of the lever body 12a that protrudes outward from the elastic holding portion 7a of the rubber boot 7 by connecting means such as screwing. Here, a sealing member 20 including an O-ring or the like is provided at a joint between a circumferential surface of the lever body 12a and the rubber boot 7 in the region where the lever body 12a protrudes from the rubber boot 7. The sealing member 20 secures water tightness in the joint between the rubber boot 7 and the lever body 12a. The other end of the lever body 12a penetrates the sphere 13 that forms part of the swinging frame 14 and is erected so as to be integrated with a substantially central part of the swinging frame 14.

The swinging frame 14 is constructed of a connecting shaft 14a, the sphere 13 integrally fixed to the connecting shaft 14a on the distal end side and a disk-shaped frame section (hereinafter referred to as “disk frame”) 14b that locks the proximal end of the plurality of bending wires 11 or the like.

The connecting shaft 14a is a hollow bar portion having a substantially circular cross-sectional shape formed so as to protrude by a predetermined height from the center of one end face of the disk frame 14b. The sphere 13 is integrally formed on a distal end side of the connecting shaft 14a. A through hole 14c is formed in the connecting shaft 14a and the sphere 13, penetrating along the major axis 3a of the operation portion 3, into which the proximal end side of the angle lever 12 is inserted.

With this configuration, the other end of the angle lever 12 is inserted through the through hole 14c of the connecting shaft 14a and the sphere 13. In this case, a stopper member 21 is provided at the opposite end of the angle lever 12, which prevents the angle lever 12 from being removed from the swinging frame 14 (the connecting shaft 14a and the sphere 13). Note that when inserted in the through hole 14c of the connecting shaft 14a and the sphere 13, the angle lever 12 is freely rotatable. With this configuration, when the angle lever 12 is tilted and the sphere 13 is moved in the sphere disposition portion 8q, the connecting shaft 14a is also tilted in the same direction together with the angle lever 12 at this time so that the disk frame 14b is swung.

A plurality of wire insertion holes 14h through which the proximal end sides of the bending wires 11 are passed and a plurality of locking concave portions 14k for locking the wire locking members 15 are formed at equal intervals in the circumferential direction (e.g., four locations at an angle interval of 90 degrees) in predetermined regions in the vicinity of the outer circumferential edge of the disk frame 14b. The plurality of wire insertion holes 14h and locking concave portions 14k are formed as many as the plurality of bending wires 11. Since the present embodiment provides a mode in which the four bending wires 11 are disposed, four wire insertion holes 14h and four locking concave portions 14k are formed as well. Here, the central axis of the locking concave portion 14k is formed to be coaxial with the central axis of the wire insertion hole 14h, and the diameter of the locking concave portion 14k is formed to be slightly larger than the diameter of the wire insertion hole 14h.

The diameter of the wire insertion hole 14h is formed to be slightly larger than the wire diameter of the bending wire 11. That is, the wire insertion hole 14h is a through hole disposed such that the bending wire 11 is loosely fitted thereinto.

The locking concave portion 14k is a concave dent formed on one side of the disk frame 14b. The wire locking member 15 fixed at a proximal end of the bending wire 11 is disposed in the locking concave portion 14k. For this reason, the diameter of the locking concave portion 14k is set to be slightly smaller than the diameter of the wire locking member 15. In this way, the locking concave portion 14k locks the wire locking member 15.

With this configuration, when the angle lever 12 is tilted and the swinging frame 14 is caused to swing around the center of the sphere 13, the bending wire 11 tows the outer circumferential edge of the disk frame 14b (wire insertion hole 14h and locking concave portion 14k). This causes the bending portion 2b to bend in a predetermined direction by a predetermined amount. In this case, the swinging frame 14 swings around the center of the sphere 13 along with the tilting operation of the angle lever 12, and at this time, the sphere 13 becomes a fulcrum of the angle lever 12.

Note that the swinging frame 14 may be configured using a frame member provided with a plurality of arm portions instead of the disk frame 14b. In this case, the number of arm portions may be configured to match the number of bending wires.

The rotation angle detection section 18 is a sensor member that converts an amount of mechanical displacement of rotation of the angle lever 12 to an electric signal, processes the signal to detect, for example, an angle of rotation or a rotating direction. For example, an absolute type rotary encoder that outputs an angle of rotation as an absolute numerical value is used for the rotation angle detection section 18.

The rotation angle detection section 18 is fixedly disposed on a substantially central part of the other side of the disk frame 14b of the swinging frame 14, that is, a surface opposite to the surface on which the connecting shaft 14a is disposed using means such as screwing. The sensor section 18b of the rotation angle detection section 18 is disposed in a region opposite to the other end distal end portion of the lever body 12a of the angle lever 12. In this way, the rotation angle detection section 18 detects an angle of rotation, rotating direction or the like around the major axis 3a of the angle lever 12. An output signal of the rotation angle detection section 18 is outputted from the operation portion 3 to the video processor section 9 through the universal cord 4.

Note that the operation portion 3 incorporates other various configuration units, but since the other configuration units are not directly related to the present invention, those units are assumed to be substantially similar to those of conventional endoscope systems and illustration and detailed description thereof will be omitted.

In the endoscope system of the present embodiment, the video processor section 9 is configured by including various image signal processing circuits such as an image generating section 9a and an image rotating section 9b which is an image rotation control section. Of these sections, the image generating section 9a is a signal processing section that receives an image signal generated by the image pickup section 17, generates a display image signal corresponding to various kinds of image signal processing, for example, various display modes or generates recording image data. On the other hand, the image rotating section 9b is a signal processing section that receives a detection signal outputted from the rotation angle detection section 18, that is, data such as a detected angle of rotation or rotating direction and performs image processing such as image rotating processing. Other various components, external devices or the like that make up the endoscope system are assumed to be substantially similar to those of conventional endoscope systems, and illustration and detailed description thereof will be omitted.

Operation when performing a bending operation using the endoscope system of the present embodiment configured in this way will be described below.

A user holds the operation portion 3 by hand and tilts the angle lever 12 in a desired direction by a desired angle. This causes a plurality of bending wires 11 to be towed in a predetermined direction via the bending operation unit 10 and the bending portion 2b of the insertion portion 2 is bent in a predetermined direction by a predetermined amount.

In this case, if an image being displayed on a display screen of the monitor apparatus 16 is inclined or the like and the user wants to correct the inclination of the image display, the user rotates the angle lever 12 around the major axis 3a, and can thereby correct the inclination of the display image.

A rotating operation of the angle lever 12 around the major axis 3a in this case may be performed in accordance with the inclination direction or inclination angle of the display image on the monitor apparatus 16. That is, the inclination direction of the display image is corrected by rotating the angle lever 12 in a direction opposite to the direction in which the image being displayed is inclined. Furthermore, the inclination angle of the display image is corrected by adjusting the amount of rotation of the angle lever 12 in accordance with the inclination angle of the image being displayed. That is, the amount of rotation of the angle lever 12 is increased as the inclination angle of the image becomes acuter.

The rotating operation of the angle lever 12 around the major axis 3a is immediately detected by the rotation angle detection section 18 and transmitted to the video processor section 9. In response to this, the video processor section 9 performs image rotating processing using the image rotating section 9b. As a result, an image subjected to the rotating processing is immediately displayed on the monitor apparatus 16. Therefore, the user can make fine adjustment to correct the inclination of the image while viewing the display screen of the monitor apparatus 16.

In this case, the rotating direction of the angle lever 12 is set so that the user can intuitively operate the angle lever 12 to correct the inclination of the image in accordance with the inclination correction direction of the image. More specifically, when the inclination of a horizontal line and a vertical line of the image on the display screen of the monitor apparatus 16 is downward to the right, for example, the inclination correction operation can be done by rotating the angle lever 12 counterclockwise. Adjusting the degree of correction of the angle of inclination of the image can be done in accordance with the amount of rotation of the angle lever 12. The other operations are substantially similar to those of conventional endoscope systems.

As described above, the first embodiment provides an endoscope system including the endoscope 1 provided with the bending operation unit 10 that tows the bending wire 11 to cause the bending portion 2b to bend by tilting the angle lever 12, in which the angle lever 12 is configured to be freely rotatable, the rotation angle detection section 18 for detecting rotation of the angle lever 12 is provided, an image signal generated by the image pickup section 17 is subjected to predetermined electrical image rotating processing based on an output of the rotation angle detection section 18 so as to correct an inclination of an image displayed on the monitor apparatus 16.

The endoscope system of the present embodiment in this configuration can easily and optionally correct the inclination along with a bending operation on an image displayed on the image display apparatus (monitor apparatus 16) in response to an operation based on the user's will.

In that case, the endoscope system is configured so that without separately providing any operation member for correcting an image inclination, the angle lever 12 which is an operation member provided in the conventional endoscope system and a bending operation member for performing a bending operation is configured to be freely rotatable, and an operation of rotating the angle lever 12 is performed, to thereby correct the inclination of the image. Therefore, it is possible to implement a desired image inclination correction function while avoiding enlargement of the operation portion 3 or complication of operability without additionally disposing the operation member or the like.

Furthermore, the operation for correcting an image inclination along with the bending operation is allowed to be done by the angle lever 12 for performing a bending operation and the operation for correcting an image inclination is set so that the rotating direction of the angle lever 12 matches the image inclination correction direction, and it is thereby possible to secure intuitive operability.

The endoscope system is configured so that rotation of the angle lever 12 is detected by the rotation angle detection section 18 and an image signal generated by the image pickup section 17 is subjected to electrical image rotating processing based on the detection result, and it is thereby possible to eliminate the necessity for a mechanism of mechanically rotating the image pickup section 17 or the like and easily implement a desired image rotating function even using a small-diameter endoscope 1.

### Second Embodiment

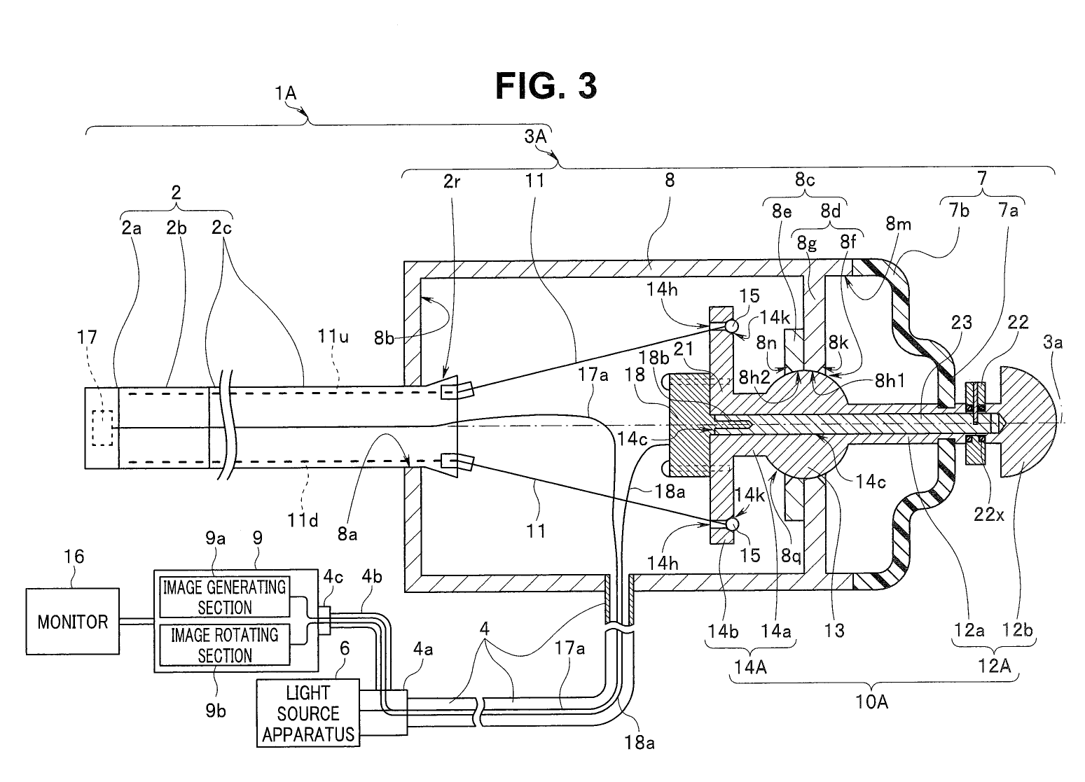

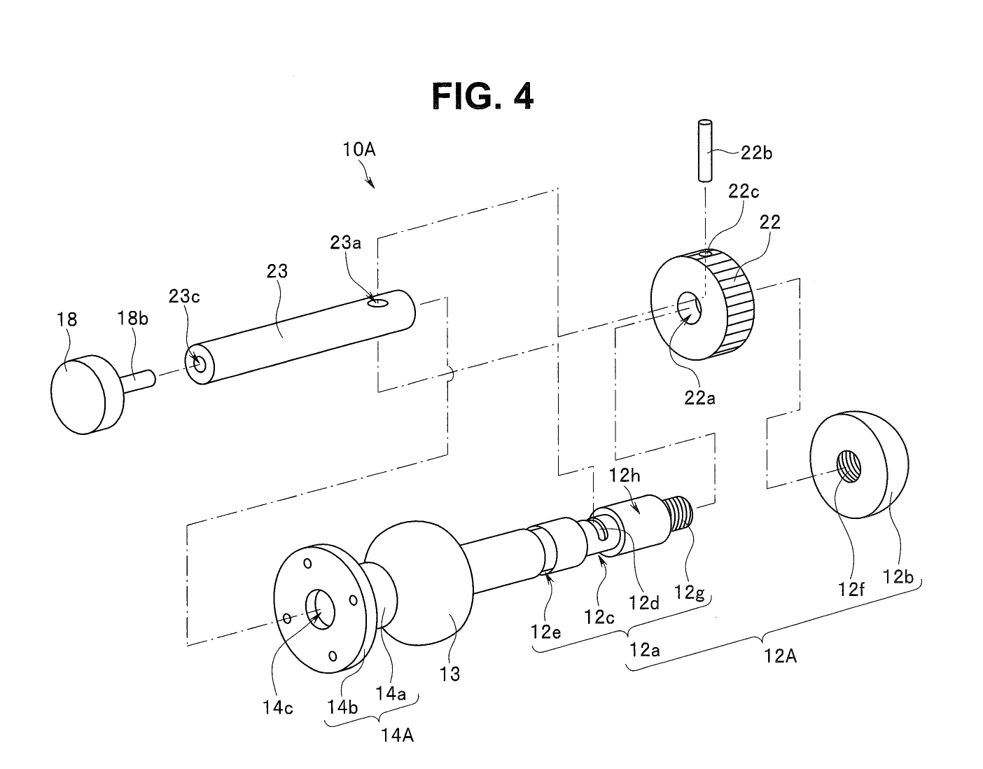

Next, an endoscope system according to a second embodiment of the present invention will be described below using FIG. 3 and FIG. 4. The above-described endoscope system of the first embodiment is configured such that the angle lever 12 in the bending operation unit 10 of the endoscope 1 is configured to be freely rotatable around the axis, and an image inclination is corrected by rotating the angle lever 12. The configuration of the present embodiment is basically substantially similar to that of the first embodiment with only the exception that a configuration of a bending operation unit 10A of an endoscope 1A is slightly different. Therefore, components similar to those of the aforementioned first embodiment are assigned the same reference numerals, detailed description thereof is omitted and only different parts will be described below.

FIG. 3 is a configuration diagram illustrating a schematic configuration of an endoscope system and a bending operation unit inside an operation portion according to the second embodiment of the present invention. FIG. 4 is an enlarged exploded perspective view of main parts with some of components of the bending operation unit in the endoscope system in FIG. 3 extracted and shown in exploded form.

As shown in FIG. 3, a schematic configuration of the endoscope system of the present embodiment is substantially similar to the aforementioned first embodiment in that it is configured by including an endoscope 1A made up of the insertion portion 2, an operation portion 3A and the universal cord 4, and various external devices connected to the endoscope 1A (e.g., video processor section 9 to which the light source apparatus 6 and the monitor apparatus 16 are connected).

Here, in the present embodiment, the configuration of the bending operation unit 10A provided in the operation portion 3A of the endoscope 1A is slightly different from the bending operation unit 10 of the aforementioned first embodiment.

In the present embodiment, the bending operation unit 10A is mainly configured by including a body section in which a plurality of bending wires 11, an angle lever 12A, a sphere 13 and a swinging frame 14A are formed into one unit as shown in FIG. 3, a correction dial 22 for an image inclination correction operation, the rotation angle detection section 18 that detects an angle of rotation of the correction dial 22 and a dial connecting shaft 23 that is interposed between the correction dial 22 and the rotation angle detection section 18 to transmit rotation of the correction dial 22 to the rotation angle detection section 18.

The connection structure between the plurality of bending wires 11 and the swinging frame 14A is completely the same as that of the aforementioned first embodiment.

In the bending operation unit 10A of the present embodiment, the angle lever 12A which is a bending operation member, the sphere 13 disposed in the sphere disposition portion 8q of the bending mechanism attaching portion 8c in the inner frame 8 and the swinging frame 14A are formed into one unit.

As shown in FIG. 4, the angle lever 12A is constructed of the lever body 12a and the finger hooking portion 12b. In the angle lever 12A, the lever body 12a and the finger hooking portion 12b are freely detachably formed as shown in FIG. 4, and both parts are configured into one body by screwing together a male thread 12g of the lever body 12a and a female thread 12f of the finger hooking portion 12b.

Here, in the region where the lever body 12a protrudes from the rubber boot 7, a joint between a circumferential surface of the lever body 12a and the rubber boot 7 is bonded using, for example, an adhesive. The relevant region in the aforementioned first embodiment is configured by providing the sealing member 20 to secure rotation and tilting of the angle lever 12. In the present embodiment, since the angle lever 12 is only tilted without rotation, the sealing member 20 is eliminated, the rubber boot 7 and the lever body 12a are bonded and fixed, and water tightness of the joint is thereby secured.

Furthermore, in the angle lever 12A, a circumferential groove 12c for disposing the correction dial 22 is formed on an outer circumferential face between the region where the lever body 12a protrudes from the rubber boot 7, which is a joint between the lever body 12a and the rubber boot 7 and the finger hooking portion 12b. A rotation restricting groove 12d is drilled in this circumferential groove 12c in a circumferential direction by a predetermined length for restricting rotation of the correction dial 22.

The other end of the angle lever 12A is formed integrally with the sphere 13. The sphere 13 is formed integrally with the connecting shaft 14a of the swinging frame 14A. The connecting shaft 14a is integrally erected at a substantially central part of the disk frame 14b. Thus, the through hole 14c that penetrates along the major axis 3a is formed in the angle lever 12A, the sphere 13, the connecting shaft 14a and the disk frame 14b.

The hollow cylindrical or solid columnar dial connecting shaft 23 is inserted through the through hole 14c so as to be freely rotatable around the axis. A side hole 23a for pin insertion is drilled in the vicinity of the distal end portion of the dial connecting shaft 23 in a direction orthogonal to a major axis thereof. The side hole 23a is a hole into which a bar-like pin 22b is fitted when the dial connecting shaft 23 is inserted through the through hole 14c. Furthermore, a sensor disposition hole 23c which is framed in a direction along the major axis and in which a sensor section 18b of the rotation angle detection section 18 is disposed is formed on a rear end face of the dial connecting shaft 23.

The correction dial 22 is an operation member for performing an image inclination correction operation and is a rotating operation member formed into, for example, a substantially disk-like shape. The correction dial 22 is disposed so as to be freely rotatable around the major axis 3a of the lever body 12a in a predetermined region (circumferential groove 12c) of the lever body 12a of the angle lever 12A.

A rotation central hole 22a that penetrates in a direction along the major axis 3a when the correction dial 22 is attached to the lever body 12a is formed in a substantially central part of the correction dial 22. In addition, a side hole 22c is formed which penetrates in a diameter direction from the lateral circumferential surface of the correction dial 22 to the rotation central hole 22a. A bar-like pin 22b is fitted into the side hole 22c. The bar-like pin 22b is a fixing member that fixes the correction dial 22 to the dial connecting shaft 23 via the lever body 12a.

Note that a sealing member 22x such as an O-ring is disposed between the correction dial 22 and the circumferential groove 12c of the lever body 12a. Water tightness between the correction dial 22 and the circumferential groove 12c of the lever body 12a is secured in this way.

Here, the bending operation unit 10A of the present embodiment is assembled as follows. That is, the dial connecting shaft 23 is inserted through the through hole 14c first. In this case, the side hole 23a of the dial connecting shaft 23 is disposed at a position opposing the circumferential groove 12c of the lever body 12a.

Next, the correction dial 22 is attached to the lever body 12a. For that purpose, a distal end portion 12h of the lever body 12a is inserted through the rotation central hole 22a of the correction dial 22 and the correction dial 22 is disposed at a position corresponding to the circumferential groove 12c of the lever body 12a first. For this purpose, the diameter of the distal end portion 12h of the lever body 12a is formed to be slightly smaller than the diameter of the rotation central hole 22a of the correction dial 22. In this case, the side hole 22c of the correction dial 22 is disposed at a position opposing the circumferential groove 12c of the lever body 12a.

In this condition, the bar-like pin 22b is inserted in a diameter direction toward the rotation central hole 22a from the side hole 22c of the correction dial 22. The bar-like pin 22b penetrates the side hole 22c, then penetrates the rotation restricting groove 12d and is fitted into the side hole 23a. In this way, the correction dial 22 is attached integrally with the dial connecting shaft 23 via the lever body 12a. Therefore, the correction dial 22 and the dial connecting shaft 23 are rotatably disposed to the lever body 12a.

When the correction dial 22 and the dial connecting shaft 23 rotate, the bar-like pin 22b moves along the rotation restricting groove 12d of the lever body 12a. In this case, the bar-like pin 22b is movable only within a range of the rotation restricting groove 12d in the circumferential direction. Therefore, with this configuration, rotations of the correction dial 22 and the dial connecting shaft 23 are restricted by the bar-like pin 22b and the rotation restricting groove 12d.

As described above, the sensor section 18b of the rotation angle detection section 18 is disposed in the sensor disposition hole 23c of the rear end face of the dial connecting shaft 23. Therefore, with this configuration, the dial connecting shaft 23 is interposed between the correction dial 22 and the rotation angle detection section 18, and the dial connecting shaft 23 plays the role of transmitting the rotation of the correction dial 22 to the rotation angle detection section 18. The rest of the configuration is substantially similar to that of the aforementioned first embodiment.

Operation when a bending operation is performed using the endoscope system of the present embodiment configured in this way is as follows.

The user tilts the angle lever 12 while holding the operation portion 3 by hand to bend the bending portion 2b of the insertion portion 2. Operation of the bending operation unit 10A in the endoscope 1 of the endoscope system of the present embodiment is substantially similar to the operation of the bending operation unit 10 of the aforementioned first embodiment.

To correct an inclination of a display image, the correction dial 22 is rotated around the major axis 3a of the angle lever 12 in the present embodiment. In this case, the rotating operation on the correction dial 22 is similar to that of the aforementioned first embodiment in that the rotating operation is performed in accordance with the inclination direction or inclination angle of the display image of the monitor apparatus 16.

The rotating operation of the correction dial 22 around the major axis 3a is immediately detected by the rotation angle detection section 18 and transmitted to the video processor section 9. A series of operations from image rotating processing by the image rotating section 9b of the video processor section 9 in response to this to display of the image subjected to the rotating processing on the result by the monitor apparatus 16 are similar to those of the aforementioned first embodiment. The other operations are also similar to those of the aforementioned first embodiment.

As described above, according to the second embodiment, the endoscope system including the endoscope 1A provided with the bending operation unit 10A that bends the bending portion 2b by tilting the angle lever 12A and towing the bending wires 11 is configured such that the correction dial 22 rotatable around the major axis 3a of the angle lever 12 is provided, rotation of the correction dial 22 is detected by the rotation angle detection section 18, an image signal generated by the image pickup section 17 is subjected to predetermined electrical image rotating processing based on the output of the rotation angle detection section 18, and an inclination of the image displayed on the monitor apparatus 16 is thereby corrected.

With this configuration, the endoscope system of the present embodiment can obtain completely the same effects as those of the aforementioned first embodiment. In the present embodiment, the correction dial 22 is disposed to be coaxial with the angle lever 12 and rotatable around the major axis 3a of the angle lever 12, making it possible to achieve better operability.

### Third Embodiment

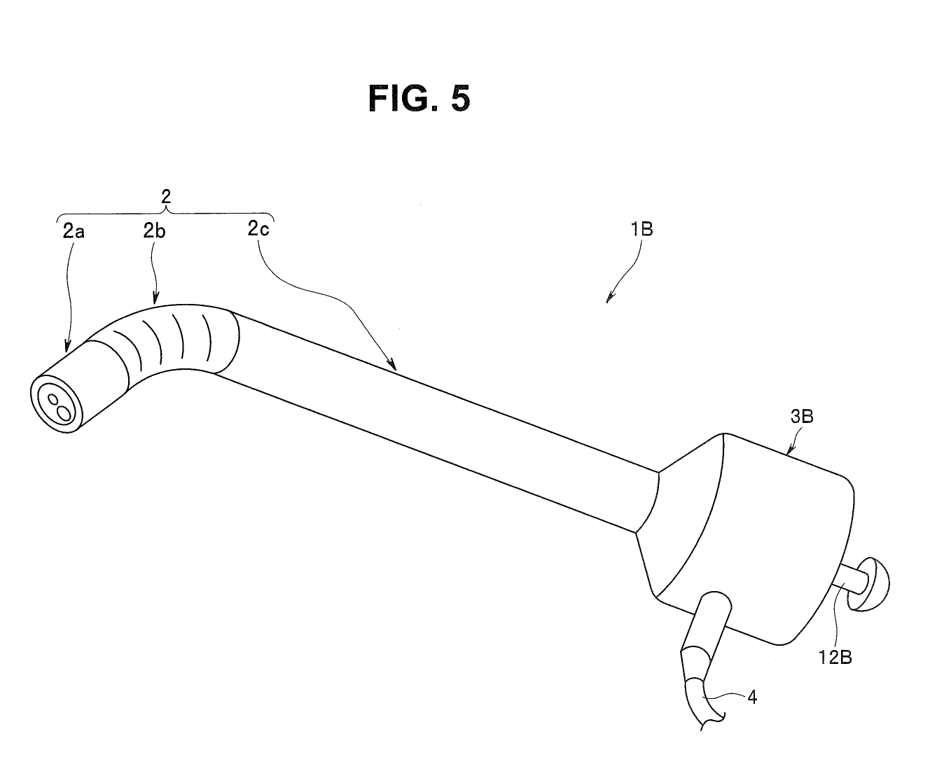

Next, an endoscope system according to a third embodiment of the present invention will be described below using FIG. 5 to FIG. 8. The endoscope system of the aforementioned first or second embodiment is configured such that the rotating operation member (angle lever 12 or correction dial 22) is provided in the bending operation unit 10 or 10A provided for the endoscope 1 or 1A, and rotation thereof is detected by the rotation angle detection section 18 so as to electrically correct an image inclination. In contrast, in the present embodiment, when the rotating operation member (angle lever 12) of a bending operation unit 10B provided in an endoscope 1B is rotated, the image pickup section 17 rotates around the insertion portion 2 and the major axis 3a of an operation portion 3B along with this rotation and an image inclination is thereby corrected.

A basic configuration of the present embodiment is substantially similar to the configuration of the aforementioned first embodiment, whereas the present embodiment is slightly different in a configuration of the bending operation unit 10B and only different in that the image pickup section 17 has a rotating mechanism. Therefore, components similar to those in the aforementioned first embodiment are assigned the same reference numerals and detailed description thereof will be omitted, and only different parts will be described below.

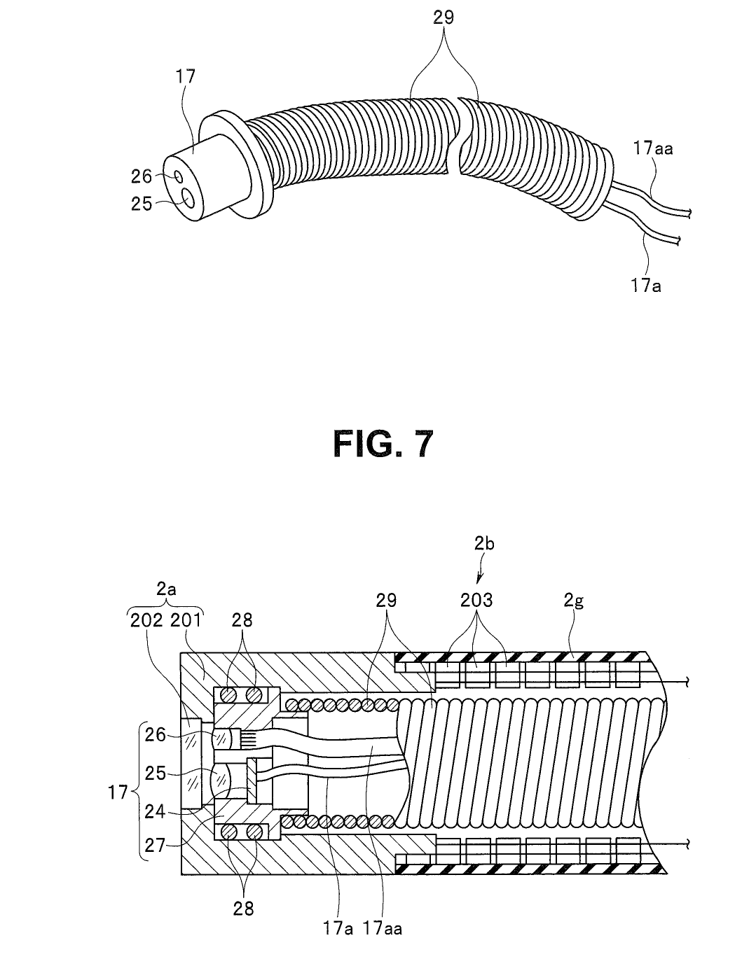

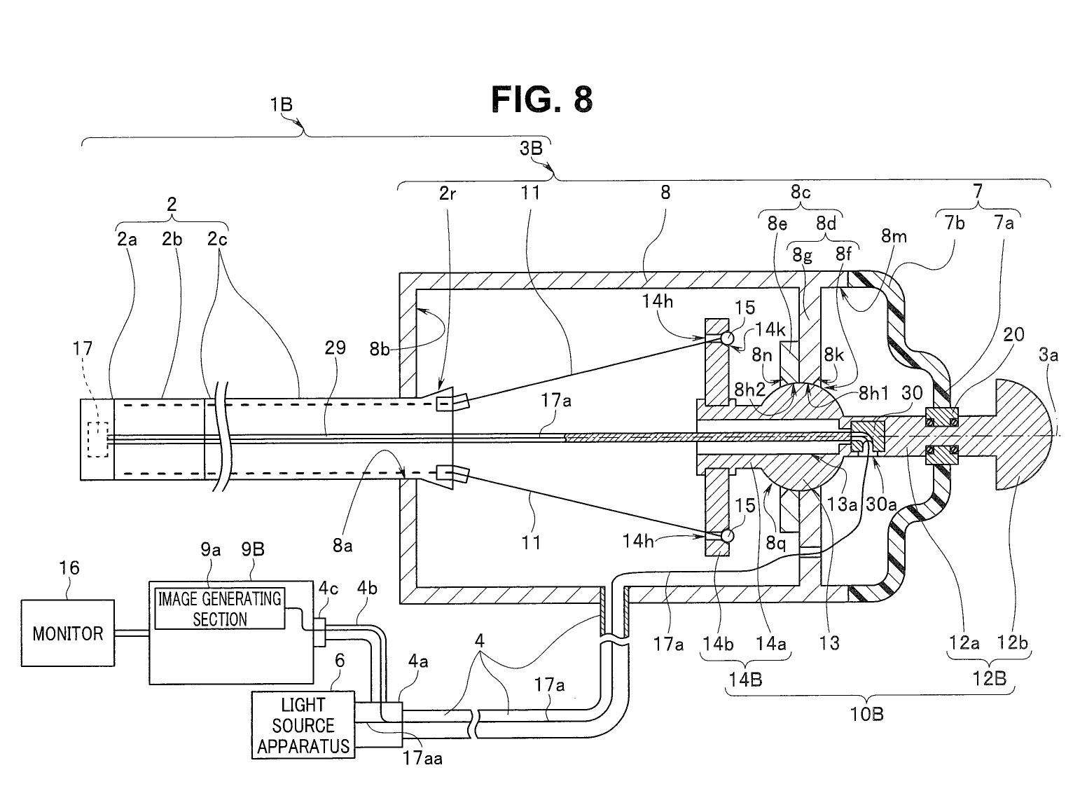

FIG. 5 is an external perspective view illustrating an entire endoscope in the endoscope system according to the third embodiment of the present invention. FIG. 6 is an external perspective view illustrating an image pickup section extracted from the endoscope of the endoscope system of the present embodiment. FIG. 7 is an enlarged cross-sectional view of main parts illustrating an inner structure of a distal end portion in the endoscope of the endoscope system of the present embodiment and a configuration of the image pickup section in particular. FIG. 8 is a configuration diagram illustrating a schematic configuration of the endoscope system of the present embodiment and a bending operation unit inside the operation portion.

The endoscope 1B in the endoscope system of the present embodiment is similar to the aforementioned embodiments in that as shown in FIG. 5, the endoscope system is configured by including the insertion portion 2 (configured by connecting the distal end portion 2a, the bending portion 2b and the rigid tube portion 2c), the operation portion 3 (including an angle lever 12B) and the universal cord 4.

The present embodiment is also similar to the aforementioned embodiments in that various external devices (light source apparatus 6 and video processor section 9B) are connected to the endoscope 1B via the universal cord 4 as shown in FIG. 8.

Note that the endoscope system of the present embodiment is different from the aforementioned embodiments in that the video processor section 9B is configured without the image rotating section 9b which is provided in the aforementioned embodiments.

In the endoscope system of the present embodiment, the distal end portion 2a of the insertion portion 2 of the endoscope 1B is configured by including a distal end frame 201 and a distal end window 202 as shown in FIG. 7. Of these components, the distal end frame 201 is a housing member making up a body of the distal end portion 2a. The distal end frame 201 is a member formed into a substantially cylindrical shape as a whole and provided with openings at both ends in the cylindrical axis direction. The image pickup section 17 is held to be freely rotatable inside the distal end frame 201 (detailed configuration will be described later). Furthermore, the distal end window 202 is disposed so as to cover one surface (front side) of the distal end frame 201 in the cylindrical axis direction and is made of a circular transparent resin member, for example.

The image pickup section 17 is disposed inside the distal end frame 201 of the distal end portion 2a. As shown in FIG. 6 and FIG. 7, the image pickup section 17 is configured by mainly including an image pickup device 24, an image pickup optical system 25, an illumination optical system 26, an image pickup section body 27, a signal cable 17a and an illumination light guide 17aa or the like.

The image pickup optical system 25 is an optical member that forms an optical image of an object to be observed. The image pickup device 24 is an electronic part that receives light of an optical image formed by the image pickup optical system 25, photoelectrically converts the optical image and generates an electronic image signal. The signal cable 17a to transmit a control signal for controlling the image pickup device 24 and an image signal generated by the image pickup device 24 extends from the image pickup device 24. This signal cable 17a passes through the insertion portion 2, the operation portion 3 and the universal cord 4 of the endoscope 1B, passes through the cable 4b via the LG connector 4a and is connected to the signal connector 4c. Thus, when the signal connector 4c is connected to the video processor section 9B, a control signal is transmitted from the video processor section 9B to the image pickup device 24 and an output signal of the image pickup device 24 is transmitted to the video processor section 9B.

The illumination optical system 26 is an optical member for emitting illuminating light transmitted from the light source apparatus 6 to the illumination light guide 17aa toward an object to be observed on the front side of the distal end portion 2a of the insertion portion 2 of the endoscope 1B.

The illumination light guide 17aa is a light transmission cable that transmits illuminating light emitted from the light source apparatus 6 to a distal end side of the insertion portion 2 of the endoscope 1B. The illumination light guide 17aa is inserted through the insertion portion 2, the operation portion 3 and the universal cord 4 of the endoscope 1, one end of which is disposed behind the illumination optical system 26 and the other end of which is connected to the LG connector 4a. Thus, when the LG connector 4a is connected to the light source apparatus 6, illuminating light from the light source apparatus 6 is transmitted to the illumination optical system 26 and emitted forward by the illumination optical system 26.

The image pickup section body 27 is a housing member that fixes each component making up the image pickup section 17 to a predetermined region therein and holds the image pickup section 17 so as to be freely rotatable inside the distal end frame 201 of the distal end portion 2a. Thus, when the image pickup section body 27 is inserted in the distal end frame 201, a roller member 28 that holds the image pickup section body 27 so as to be freely rotatable around the axis of the insertion portion 2 in the insertion direction is disposed between an outer circumferential face of the image pickup section body 27 and an inner surface of the distal end frame 201.

As described above, one end of a flexible shaft 29 is connected and fixed behind the image pickup section 17. The flexible shaft 29 is a flexible tube member that covers and allows the signal cable 17a and the illumination light guide 17aa to pass therethrough. Thus, the flexible shaft 29 passes through the insertion portion 2 and the other end thereof extends to the inside of the operation portion 3. Here, a rear end member 30 is fixed to the other end of the flexible shaft 29 as shown in FIG. 8. The rear end member 30 is fixed to the inside of the angle lever 12B (detailed configuration will be described later).

The bending portion 2b is mainly constructed of a bending piece unit 203 (see FIG. 7) configured to be freely bendable in four directions of up, down, right and left, for example, with respect to the insertion axis direction and bending rubber 29 that covers an outer face of the bending piece unit 203 or the like. Note that the configuration of the bending portion 2b itself is assumed to have a configuration similar to that of a conventional endoscope and detailed description thereof will be omitted.

Next, in the endoscope system of the present embodiment, a configuration of the bending operation unit 10B provided in the operation portion 3B of the endoscope 1B will be described below using mainly FIG. 8.

A basic configuration of the bending operation unit 10B of the present embodiment is substantially similar to that of the aforementioned first embodiment. In the present embodiment, the bending operation unit 10B is mainly constructed of an angle lever 12B, the sphere 13, and a swinging frame 14B made up of the connecting shaft 14a and the disk frame 14b. In the bending operation unit 10B, the angle lever 12B, the sphere 13 and the connecting shaft 14a are integrated into a single unit. One end of the connecting shaft 14a is erected integrally with a substantially central part of the disk frame 14b. Here, the disk frame 14b restricts motion of the connecting shaft 14a in a direction along the major axis 3a and is connected to the connecting shaft 14a so as to be rotatable around the major axis 3a. An insertion hole 13a that secures a predetermined space region is formed inside the sphere 13 and the connecting shaft 14a. The flexible shaft 29 that extends from the image pickup section 17 is inserted through the insertion hole 13a. The rear end member 30 of the flexible shaft 29 is fixedly held in a predetermined region inside the angle lever 12B.

An insertion hole 30a is formed in the rear end member 30. The insertion hole 30a is formed in a region corresponding to a hole formed on a circumferential surface of the lever body 12a of the angle lever 12B. The signal cable 17a and the illumination light guide 17aa extend from the insertion hole 30a. Note that the signal cable 17a and the illumination light guide 17aa are connected from the operation portion 3 to the video processor section 9B and the light source apparatus 6 via the universal cord 4. The rest of the configuration is substantially similar to that of the aforementioned first embodiment.

In the endoscope system of the present embodiment configured as described above, the bending portion 2b is bent through a tilting operation of the angle lever 12B as in the case of the aforementioned first embodiment. When the angle lever 12B is rotated, the rear end member 30 fixed to the angle lever 12B rotates in the same direction and the image pickup section 17 rotates in the same direction via the flexible shaft 29, that is, around the axis of the insertion direction of the insertion portion 2, in the same direction as the direction in which the correction dial 22 is rotated. When the image pickup section 17 rotates, the image on the display screen of the monitor apparatus 16 also rotates. Thus, an image inclination is corrected. In this case, the rotating direction of the angle lever 12B is set to match the rotating direction of the image pickup section 17. Therefore, the user can intuitively correct an inclination of the image by rotating the angle lever 12B while viewing the display screen of the monitor apparatus 16. It is also possible to make fine adjustment of the amount of correction in that case.

As described above, according to the third embodiment, the endoscope system including the endoscope 1B provided with the bending operation unit 10B that bends the bending portion 2b by tilting the angle lever 12B and towing the bending wire 11 is configured such that the angle lever 12B is configured to be freely rotatable and the image pickup section 17 also rotates in the same direction along with the rotating operation of the angle lever 12B. This configuration makes it possible to easily correct an inclination of an image displayed on the monitor apparatus 16 by only rotating the angle lever 12B.

### Fourth Embodiment

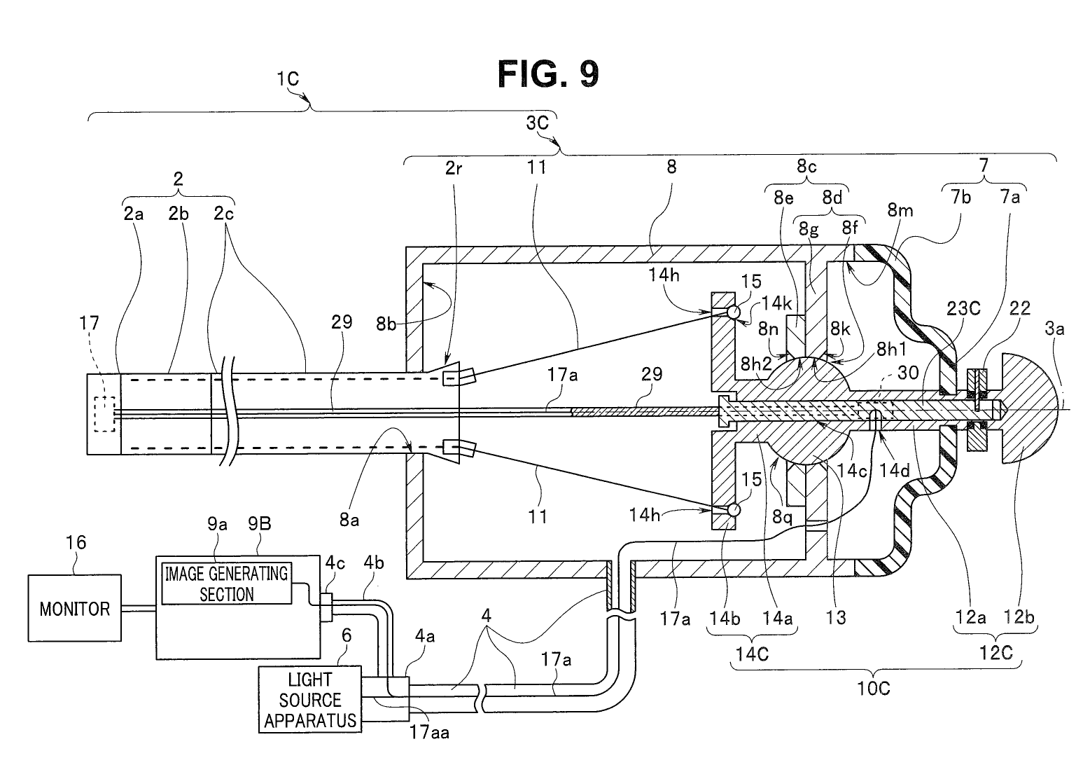

Next, an endoscope system according to a fourth embodiment of the present invention will be described below using FIG. 9. A configuration of the present embodiment is basically substantially similar to the configuration of the aforementioned third embodiment but is different in that the operation member for rotating the image pickup section 17 for correcting an image inclination is configured of a correction dial (22) substantially similar to the second embodiment. Therefore, components similar to those of the aforementioned third embodiment are assigned the same reference numerals, description thereof is omitted and only different parts will be described below.

FIG. 9 is a configuration diagram illustrating a schematic configuration of an endoscope system and a bending operation unit inside an operation portion according to a fourth embodiment of the present invention.

As shown in FIG. 9, the schematic configuration of the endoscope system of the present embodiment is substantially similar to that of the aforementioned third embodiment in that the endoscope system is configured by including an endoscope 1C made up of the insertion portion 2 and an operation portion 3C and the universal cord 4 and various external devices connected to the endoscope 1C (e.g., video processor section 9B to which the light source apparatus 6 and the monitor apparatus 16 are connected).

Here, in the present embodiment, a configuration of a bending operation unit 10C provided in the operation portion 3C of the endoscope 1C is slightly different from the bending operation unit 10B in the aforementioned third embodiment.

In the present embodiment, the bending operation unit 10C is mainly constructed of a body section in which a plurality of bending wires 11, an angle lever 12C, the sphere 13 and a swinging frame 14C are integrated into a single unit, a correction dial 22 for an image inclination correction operation, and a dial connecting shaft 23C that transmits rotation of the correction dial 22 to the image pickup section 17 as shown in FIG. 9.

The connection structure between the plurality of bending wires 11 and the swinging frame 14C is completely the same as that of the aforementioned embodiments.

In the bending operation unit 10C of the present embodiment, the angle lever 12C which is a bending operation member, the sphere 13 disposed in the sphere disposition portion 8q of the bending mechanism attaching portion 8c in the inner frame 8 and the swinging frame 14C are integrated into a single unit.

As shown in FIG. 9, the angle lever 12C is made up of the lever body 12a and the finger hooking portion 12b, and has a configuration substantially similar to that of the angle lever 12A applied in the aforementioned second embodiment. That is, the rubber boot 7 and the lever body 12a are joined together to secure water tightness of the joint.

As in the case of the angle lever 12C in the aforementioned second embodiment, the angle lever 12C, the sphere 13, the connecting shaft 14a and the disk frame 14b are integrated into a single unit and a through hole 14c that penetrates along the major axis 3a is formed in the angle lever 12C. A hollow cylindrical or solid columnar dial connecting shaft 23C is inserted into the through hole 14c so as to be freely rotatable around the axis. The rear end member 30 of the flexible shaft 29 that extends from the image pickup section 17 is fixed to the dial connecting shaft 23C. The correction dial 22 is attached to the dial connecting shaft 23C so as to be rotatable integrally therewith via the lever body 12a. The attaching structure of the correction dial 22 is substantially similar to that of the aforementioned second embodiment (see FIG. 3 and FIG. 4). The rest of the configuration is substantially similar to that of the aforementioned third embodiment.

Operation when performing a bending operation using the endoscope system of the present embodiment configured as described above is as follows.

The user holds the operation portion 3 by hand and tilts the angle lever 12C to bend the bending portion 2b of the insertion portion 2. Operation of the bending operation unit 10C in the endoscope 1 of the endoscope system of the present embodiment is substantially similar to the operation of the bending operation unit of the aforementioned embodiments.

In the present embodiment, to correct an inclination of a display image, the correction dial 22 is rotated around the major axis 3a of the angle lever 12C. The operation of the correction dial 22 in this case is similar to that of the aforementioned second embodiment.

The rotating operation of the correction dial 22 around the major axis 3a is performed by rotating the dial connecting shaft 23C in the same direction. In this way, the rear end member 30 fixed to the dial connecting shaft 23C also rotates in the same direction and the image pickup section 17 rotates via the flexible shaft 29 in the same direction, that is, around the axis of the insertion direction of the insertion portion 2 in the same direction as the direction in which the correction dial 22 is rotated.

When the image pickup section 17 rotates, the image on the display screen of the monitor apparatus 16 also rotates and the inclination of the image is thereby corrected. In this case, the rotating direction of the correction dial 22 is set to match the rotating direction of the image pickup section 17. Therefore, by rotating the correction dial 22 while viewing the display screen of the monitor apparatus 16, the user can intuitively correct an image inclination, and can also easily make fine adjustment of the amount of correction in that case.

As described above, according to the fourth embodiment, it is possible to directly rotate the image pickup section 17 around the axis in the insertion direction and easily correct the inclination of the image as in the case of the aforementioned third embodiment using a dial operation member (correction dial 22) similar to that of the aforementioned second embodiment.

Note that the present invention is not limited to the aforementioned embodiment, but it goes without saying that various modifications and applications can be made without departing from the spirit and scope of the present invention. Furthermore, the above-described embodiments include inventions in various stages and various inventions can be extracted with appropriate combinations among a plurality of components disclosed. For example, when some of components are deleted from all the components disclosed in each embodiment, if the problems to be solved by the invention can be solved and the effects of the invention can be obtained, the configuration from which the components are deleted can be extracted as the invention. Furthermore, components belonging to different embodiments may also be combined as appropriate.

The present invention is applicable not only to endoscope control apparatuses in the medical field but also to endoscope control apparatuses in the industrial field.

What is claimed is:

1. An endoscope system comprising:
an image pickup section that photoelectrically converts an optical image to generate an electronic image signal;
an insertion portion that incorporates the image pickup section, comprises a bendable bending portion and is inserted into a subject;
a lever portion that is swingable in a direction orthogonal to a central axis, held so as to be rotatable around the central axis and swung to thereby bend the bending portion; and
a video processor section that receives an image signal generated by the image pickup section, generates a display image signal, performs rotating image signal processing on the display image signal in accordance with a rotation of the lever portion and outputs a processing result to an image display apparatus.

2. The endoscope system according to claim 1, wherein the lever portion is provided in an operation portion connected to a proximal end side of the insertion portion.

3. The endoscope system according to claim 2, wherein the lever portion is connected to the bending portion via a towing member.

4. The endoscope system according to claim 3, further comprising a rotation angle detection section that detects an angle of rotation of the lever portion, wherein the rotation angle detection section is provided in the operation portion.

5. The endoscope system according to claim 4, wherein the video processor section comprises an image rotation control section that receives rotation angle information detected by the rotation angle detection section, performs the rotating image signal processing based on the rotation angle information and outputs the processing result to the image display apparatus.

6. The endoscope system according to claim 3, wherein the image pickup section is connected to the lever portion, held so as to be rotatable around a longitudinal axis of the insertion portion and rotates along with the rotation of the lever portion around the central axis.
